(19) United States
(12) Patent Application Publication
(10) Pub. No.: US 2016/0128571 A1
(43) Pub. Date: May 12, 2016

(54) SYSTEMS AND METHODS FOR TRANSMITTING GLUCOSE METER READINGS

(71) Applicant: GSM Medical Devices, Inc., Cheyenne, WY (US)

(72) Inventor: Chagai Adler, St. Louis, MO (US)

(21) Appl. No.: 14/937,807

(22) Filed: Nov. 10, 2015

Related U.S. Application Data
(60) Provisional application No. 62/077,516, filed on Nov. 10, 2014.

(51) Int. Cl. A61B 5/00; A61B 5/15; A61B 5/145

(52) U.S. Cl. CPC A61B 5/0022; A61B 5/14532; A61B 5/150358; A61B 5/72; A61B 5/742; A61B 5/0004; A61B 2560/0475

(57) ABSTRACT

Devices, methods, and systems for transmitting, via USSD messages, blood glucose readings from a glucometer over a GSM network to a database storing blood glucose readings. The database is configured to transmit electronic messages including the blood glucose readings received from the glucometer to one or more personal electronic devices of one or more predetermined recipients.

This application claims the benefit of U.S. Provisional Application No. 62/077,516, filed Nov. 10, 2014, for SYSTEMS AND METHODS FOR ANALYZING AND TRANSMITTING GLUCOSE METER READINGS, which is incorporated in its entirety herein by reference.

### TECHNICAL FIELD

The present disclosure relates generally to transmission of data over a network, and more specifically, to devices, systems, and methods for transmitting blood glucose data captured by a glucose meter over a telecommunication network.

### BACKGROUND

Glucose meters or glucometers are used by people with type 1, type 2, and gestational diabetes to measure their sugar levels. Conventional glucometers accept a test strip, and based on a blood sample (e.g., drop of blood) applied to the test strip, to generate a blood glucose reading and display it on a display screen (e.g., LCD, LED) to a user. Conventional glucometers may include internal hard drives that store blood glucose readings and may be connected to a computer to transfer the blood glucose readings to a personal computer such that the blood glucose readings may be formatted into graphs indicating the user's trends in maintaining his or her sugar levels.

Conventional glucometers generally do not connect to the internet and/or to wireless networks and the blood glucose readings are typically not sent to people other than the user whose blood glucose is being measured by the glucometer. A disadvantage of such conventional glucometers is that for users such as young children and elderly persons who may not be checking their blood sugars regularly, their respective parents and caretakers, when not physically present to observe the child or elderly person to check their blood sugar, are not able to effectively ensure that the young child or elderly person regularly checks his or her blood sugar.

### SUMMARY

In one embodiment, an electronic blood glucose measurement device includes a blood glucose module including a microprocessor, a global system for mobile (GSM) module in communication with the blood glucose module; and a Subscriber Identification Module (SIM) in communication with the GSM module. The GSM module is configured to receive a blood glucose reading from the blood glucose module, connect to a network via the SIM; and transmit an Unstructured Supplementary Service Data (USSD) message including the blood glucose reading over a network to a server in communication with a database configured to store the blood glucose reading.

The electronic blood glucose measurement device may include an input configured to receive a test strip and the blood glucose module may be configured to generate a blood glucose reading based on a blood sample applied to the test strip and to send the generated blood glucose reading to the GSM module in response to removal of the test strip from the input.

One or both of the microprocessor of the blood glucose module and the GSM module may be configured to format the at least on blood glucose reading in order to generate the USSD message.

The SIM of the electronic blood glucose measurement device may be a physical SIM card or a virtual SIM.

In one approach, the USSD message does not exceed 80 characters.

In an embodiment, the at least one blood glucose reading in the USSD message includes a reading representing a last blood glucose reading successfully transmitted to the server and the blood glucose reading. It may also have readings not transmitted before.

In one embodiment, the electronic blood glucose measurement device includes a non-volatile memory and an indication pointer configured to point to an address in the non-volatile memory representing a last blood glucose reading successfully transmitted to the server. The electronic blood glucose measurement device may be configured to receive a confirmation from the server indicating that the USSD message including the blood glucose reading transmitted from the device was received by the server, and to update the indication pointer to point to an address in the non-volatile memory representing the blood glucose reading successfully transmitted to the server.

The electronic blood glucose measurement device may be configured, upon receiving an indication that the USSD message was not successfully transmitted from the device to the server, to store the blood glucose reading in the non-volatile memory, and to transmit another USSD message including the stored blood glucose reading after another blood glucose reading is generated by the blood glucose module when a connection to the server becomes available.

In some embodiments, the electronic blood glucose measurement device may be configured to receive the confirmation from the server in a form of an USSD message.

In one embodiment, a system for transmitting blood glucose readings includes an electronic blood glucose measurement device and a server in communication with the electronic blood glucose measurement device over a global system for mobile communications (GSM) network. The electronic blood glucose measurement device is configured to generate a blood glucose reading and transmit a USSD message including the blood glucose reading to the server over the GSM network.

The system may include a home location register (HLR) configured to receive the USSD message transmitted from the electronic blood glucose measurement device, and send it to the USSD server. The USSD server may be configured to receive a command representing the USSD message converted to an Extensible Markup Language (XML) command, and to transmit the XML command to the application server.

The application server may be configured to transmit the XML command to an IP address of a website-based database. The database may be configured to process the XML command and store the received blood glucose reading, to generate a reply XML command, and transmit the reply XML command to the application server. The application server may be configured to transmit the reply XML command received from the database to the USSD server. The USSD server convert the XML message to USSD message and send it to the HLR. The electronic blood glucose measurement device may be configured to receive a USSD message representing the reply from the database. The database may be configured to send a text message including the blood glucose reading to a mobile device of one or more predetermined recipients.

In one embodiment, the electronic blood glucose measurement device includes a visual display configured to display a message indicating whether the USSD message was successfully transmitted from the electronic blood glucose measurement device to the web site and successfully stored in the database.

The electronic blood glucose measurement device may further include a GSM module and a SIM, and wherein the electronic blood glucose measurement device is configured to transmit the USSD message to the server from the GSM module via the SIM.

In one embodiment, a method of transmitting a blood glucose reading includes: receiving a blood sample at an electronic blood glucose measurement device and outputting a blood glucose reading at the electronic blood glucose measurement device based on the blood sample; generating a USSD message including the blood glucose reading at the electronic blood glucose measurement device; transmitting the USSD message over a global system for mobile (GSM) network to a server in communication with a database; and storing the blood glucose reading received in the USSD message on the database and acknowledging successful recording of the blood glucose reading to the electronic blood glucose measurement device. The method may further include transmitting a message including the blood glucose reading stored in the database to a mobile device of one or more predetermined recipients.

The devices, systems, and methods described in this application advantageously utilize USSD messages to transmit data, providing a faster, simpler, more secure, and more cost-efficient communication platform to send messages as compared to conventional messenger platforms such as short message service (SMS). Further advantages will be appreciated by those of ordinary skill in the art with reference to the following drawings, detailed description, and claims.

### BRIEF DESCRIPTION OF THE DRAWINGS

Disclosed herein are embodiments of systems, apparatuses and methods pertaining to transmission of glucose measurement readings over a network. This description includes drawings, wherein:

FIG. 1 is a diagram of a system of transmitting a blood glucose reading over a network in accordance with some embodiments.

FIG. 2 is a functional diagram of an electronic blood glucose measurement device in accordance with several embodiments.

FIG. 3 is a flow chart diagram of a process of transmitting a blood glucose reading over a network in accordance with some embodiments.

Elements in the figures are illustrated for simplicity and clarity and have not been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, this disclosure describes devices, systems, and methods for transmitting, via USSD messages, blood glucose readings from a glucometer over a GSM network to a database storing blood glucose readings, which may then send electronic messages including the blood glucose readings received from the glucometer to one or more personal electronic devices of one or more predetermined recipients.

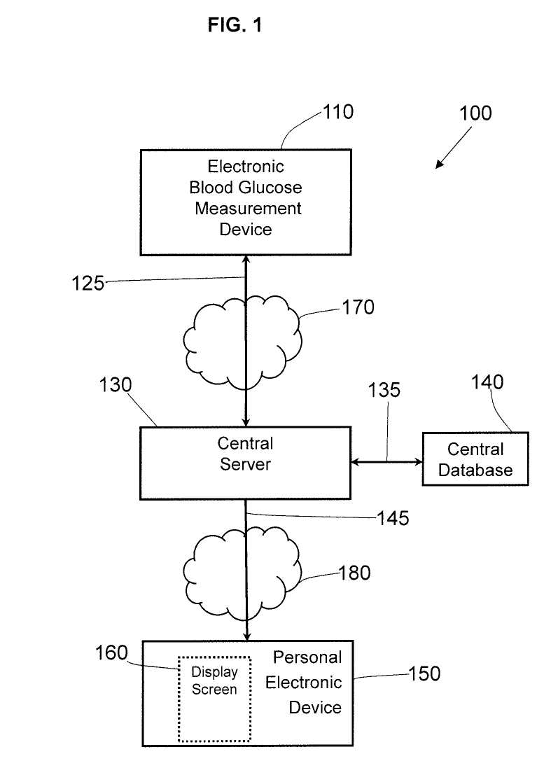

Referring to FIG. 1, one embodiment of a system 100 for transmitting blood glucose readings over a network is shown. The exemplary system 100 of FIG. 1 includes an electronic blood glucose measurement device 110, which is commonly referred to as a glucometer, which may send signals 125 over a network 170 to a central server 130 and receive signals 125 over the network 170 from the central server 130. The network 170 may be a wireless telecommunications network including but not limited to global system for mobile communications (GSM), network code division multiple access (CDMA), or the like. As used herein, the term “central server” will be understood to mean a server that receives blood glucose readings from more than one electronic blood glucose measurement device 110, and more than one “central server” may be incorporated into the system 100 of FIG. 1. The exemplary central server 130 shown in FIG. 1 is in communication (via a two-way communication channel 135) with a central database 140 configured to store blood glucose readings from multiple electronic blood glucose measurement devices 110. In the exemplary embodiment of FIG. 1, the central server 110 is optionally configured to send messages (e.g., short message service (SMS), instant messenger, electronic mail, or the like) including blood glucose readings from the central database 140 via a signal 145 over a network 180 to a mobile electronic device 150 (e.g., a mobile phone or tablet) of one or more recipients. It will be appreciated that the transmission of the blood glucose reading stored in the central database 140 to the personal electronic device 150 via the central server 130 is optional to the exemplary system 100 shown in FIG. 1. In other words, the communication channel 145 over the network 180 in FIG. 1 is optional and the system 100 may not include an active communication channel that permits the glucose readings stored in the central database 140 to be sent to the personal electronic device 150 via the central server 130. Instead, in some embodiments, one or more network devices or servers not including the central server 130 may be utilized to provide a communication channel through which the blood glucose readings may be transmitted from the central database 140 to one or more personal electronic devices 150.

FIG. 1 illustrates that the electronic data including the blood glucose readings are transmitted (via signal 125) from the electronic blood glucose measurement device 110 to the central server 130. It will be appreciated that, in some embodiments, the electronic data including the blood glucose readings may be transmitted from the electronic blood glucose measurement device 110 to the central server 130 via a home location register (HLR) and/or visitor location register (VLR).

While only one personal electronic device 150 is shown in FIG. 1, it will be appreciated that the central server 130 may be configured to send messages including the blood glucose readings from the central database 140 to more than one personal electronic device 150, and to more than one type of personal electronic devices 150. By way of example only, the personal electronic device 150 illustrated in FIG. 1 may be an electronic device, for example, a mobile phone, a tablet, a laptop, a desktop, or any other personal electronic device configured for receiving messages from the central server 130. While FIG. 1 shows that the communication channel 145 is a one-way communication channel, in some embodiments, the system 100 may be configured to permit the personal electronic device 150 to send a message via the channel 145 over the network 180 to the central server 130.

In some embodiments, the central server 130 is an electronic device including a processor-based control unit, for example, a desktop computer, a laptop computer, or any other electronic device configured for two-way communication with the electronic blood glucose measurement device 110 and/or one-way communication with another personal electronic device 150 (e.g., a smartphone). The exemplary central server 130 shown in FIG. 1 is shown to be in communication via a two communication channel 135 with a central database 140. The central database 140 may be stored, for example, on non-volatile storage media (e.g., a hard drive, flash drive, or removable optical disk) internal or external (but at the same physical site) relative to the central server 130, or may be internal or external to computing devices separate (e.g., thousands of miles away) from and remote to (e.g., thousands of miles from) the central server 130. The central database 140 may be in the form of a website having an IP address and may store electronic information including the blood glucose readings received at the central server 130 from the electronic blood glucose measurement device 110. The communication channel 135 may include, but is not limited to an electrical/data cable physically interconnecting the central server 130 and the central database 140, a wired internet connection, and a wireless internet connection.

In some embodiments, the electronic data stored in the central database 140 may be received by the central server 130 (and consequently, via the communication channel or connection 135, the central database 140), for example, as a result of a user utilizing the electronic blood glucose measurement device 110 to measure his or her blood sugar level. In some embodiments, prior to transmitting a message over the network 170, the electronic blood glucose measurement device 110 captures the local date and time from the network 170 via the GSM module 280 and includes the time and date of the blood glucose reading with the blood glucose readings included in the message transmitted over the network 170.

The electronic blood glucose measurement device 110 may also store the last blood glucose reading successfully sent over the network 170 to the central server 130, and may transmit such last successfully sent blood glucose reading again in the next USSD message as a way to enable synchronization of the central database 140 and to minimize the chances that some of the blood glucose readings do not reach the central server 130 and central database 140 due to poor GSM network connectivity. In some embodiments, the central server 130 updates the central database 140 (e.g., via the communication pathway 135) to include the new blood glucose readings received via a USSD message from the electronic blood glucose measurement device 110, but does not update the central database 140 to include the included redundant blood glucose reading reflecting the last successfully sent blood glucose reading to avoid redundancy of blood glucose readings stored in the central database 140.

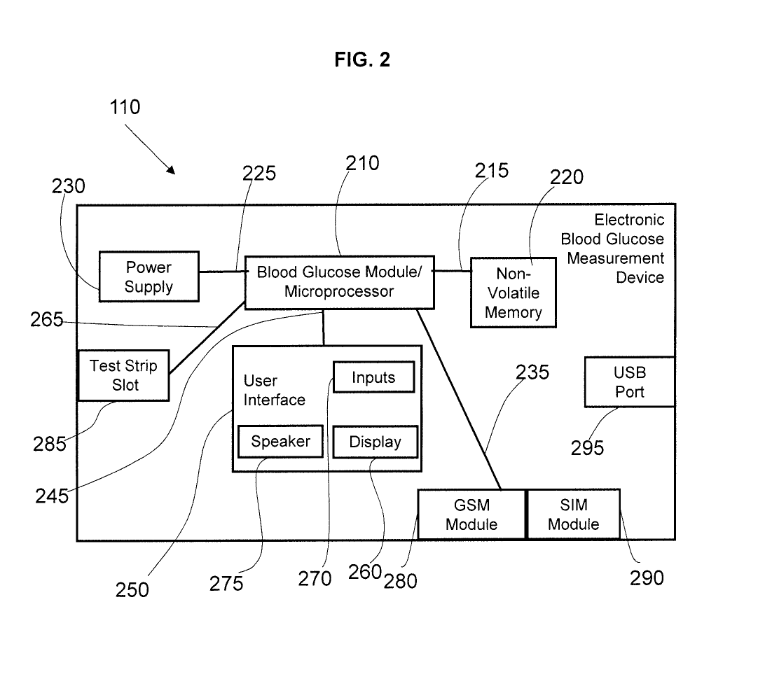

In the exemplary embodiment shown in FIG. 2, the electronic blood glucose measurement device 110 is a processor-based device and includes a blood glucose module 210 electrically coupled via a connection 215 to a non-volatile memory 220 (e.g., a hard drive or a memory card) and via a connection 225 to a power supply 230. While the exemplary electronic blood glucose measurement device 110 of FIG. 1 includes only non-volatile memory 220, it will be appreciated that the electronic blood glucose measurement device 110 may also include volatile memory (e.g., RAM). The blood glucose module 210 is configured to process blood sample data of a user and to generate corresponding blood glucose readings for the user, and can include a fixed-purpose hard-wired platform or can comprise a partially or wholly programmable platform, such as a microcontroller, microprocessor, an application specification integrated circuit, a field programmable gate array, or the like. In some embodiments, the non-volatile memory 220 of the electronic blood glucose measurement device 110 may store up to one thousand blood glucose readings per user, and may store data for one user, or for multiple (e.g., 5, 10, 20, 30, or 35) users.

In some embodiments, the blood glucose module 210 can be configured, for example, by using corresponding programming stored in the non-volatile memory 220, to carry out one or more of the steps, actions, and/or functions described herein. In some embodiments, the non-volatile memory 220 is configured non-transitorily store the computer instructions that, when executed by the blood glucose module 210, cause the blood glucose module 210 to behave as described herein. As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM)) as well as volatile memory (such as an erasable programmable read-only memory (EPROM). Accordingly, the non-volatile memory 220 and/or the blood glucose module 210 of the electronic blood glucose measurement device 110 may be referred to as a non-transitory medium or non-transitory computer readable medium.

In the exemplary embodiment shown in FIG. 2, the blood glucose module 210 of the electronic blood glucose measurement device 110 is also electrically coupled via a connection 235 to a GSM module 280 that can receive wireless signals 125 over the network 170 from the central server 130 and to send wireless signals 125 over the network 170 to the central server 130. As will be explained in more detail below, wireless signals 125 transmitted from the electronic blood glucose measurement device 110 over the network 170 to the central server 130 may include USSD messages that contain one or more blood glucose readings generated by the electronic blood glucose measurement device 110. Similarly, the wireless signals 125 transmitted from the central server 130 over the network 170 to the electronic blood glucose measurement device 110 may include acknowledgement signals in USSD messages. It will be appreciated that the acknowledgement signal sent from the central server 130 over the network 170 to the electronic blood glucose measurement device 110 may include either an acknowledgement of successful receipt of the blood glucose readings from the electronic blood glucose measurement device 110 at the central server 130 and record it on the database, or an indication of a failed transmission of the blood glucose readings from the electronic blood glucose measurement device 110 to the central server 130 or failed to record and store the reading in the database. In some embodiments, the acknowledgement signal sent from the central server 130 to the electronic blood glucose measurement device 110 is sent by a USSD message only.

In some embodiments, the connection 235 between the glucose module 210 and the GSM module is a universal asynchronous receiver/transmitter (UART) connection, but it will be appreciated that another suitable electrical connection may be used. In some embodiments, the GSM module 280 may supply power for blood glucose module 210 (e.g., via signal wires VCC and GND). In some embodiments, the GSM module 210 may send data (e.g., acknowledgement of successful transmission of blood glucose readings to central server 130) to the blood glucose module 210 via the UART connection and/or via signal wires RX and TX. In some embodiments, the blood glucose module 210 may send an interrupt signal to the GSM module 280 via a signal wire INT as a result of events, such as the reading of a blood sample by the blood glucose module 210, the plugging in and ejection of test strips into and from the test strip slot 285, or the like.

In the exemplary embodiment shown in FIG. 1, the GSM module 280 of the exemplary electronic blood glucose measurement device 110 is operatively coupled to a subscriber identify module (SIM) 290, which permits the electronic blood glucose measurement device 110 to identify itself to the network 170 and/or to the central server 130 when the GSM module 280 sends a signal 125 to the central server 130. In some embodiments, the SIM 290 may be a physical SIM card, while in other embodiments, the SIM 290 may be a virtual SIM. In some embodiments, the SIM 290 may include one or more international mobile subscriber identities (IMSIs). As used herein, an IMSI is a unique number, usually fifteen digits, associated with Global System for Mobile Communications (GSM) and Universal Mobile Telecommunications System (UMTS) network utilized by mobile phone users.

In the embodiment shown in FIG. 2, the blood glucose module 210 of the electronic blood glucose measurement device 110 is electrically coupled via a connection 245 to a user interface 250, which may include a visual display or display screen 260 (e.g., LCD, LED, TFT, or the like) and/or button input 270 that provide the user interface 250 with the ability to permit a user (i.e., person measuring his or her blood sugar levels) to manually control the electronic blood glucose measurement device 110 by inputting commands, for example, via touch-screen and/or button operation and/or voice commands. The display screen 260 can also display blood glucose readings to a user and may permit the user to see various menus, options, and/or alerts displayed by the electronic blood glucose measurement device 110.

In some embodiments, the visual display 260 of the electronic blood glucose measurement device 110 may display a message (received at the GSM module 280 from the central server 130) indicating whether the last USSD message including the blood glucose readings was successfully transmitted from the electronic blood glucose measurement device 110 to the central server 130 over the network 170. To conserve the battery life of the electronic blood glucose measurement device 110 may the visual display 260 may be configured with automatic screen shutdown triggered by a predetermined period of time of inactivity (e.g., 1 minute, 2 minutes, 3 minutes, 5 minutes, or more). In some embodiments, the electronic blood glucose measurement device 110 may include features such as automatic switch to airplane mode or sleep mode after transmitting the blood glucose readings to the central server 130 in order to conserve and extend the battery life of the electronic blood glucose measurement device 110. The user interface 250 of the electronic blood glucose measurement device 110 may also include a speaker 275 that may provide audible feedback (e.g., alerts) to the user.

The blood glucose module 210 of the exemplary electronic blood glucose measurement device 110 of FIG. 2 is also electrically coupled via a connection 265 to a test strip slot 285 configured to permit the user to insert a test strip to which a blood sample (e.g., one or more drops of blood) of the user (e.g., a person with type 1 or type 2 diabetes) may be applied after the test strip is inserted into the test strip slot 285. After a test strip is inserted into the test strip slot 285, and after the blood sample is applied to the test strip positioned in the test strip slot 285, the blood glucose module 210 is configured to receive a signal representing information about the blood sample from the test strip slot 285 via the connection 265 and to generate a blood glucose reading (e.g., numerical blood sugar value in mg/dl and/or mmol/l) based on the received blood sample information. As mentioned above, this blood glucose reading may be displayed on the electronic visual display 260 of the electronic blood glucose measurement device 110. As will be discussed in more detail below, in some embodiments, the blood glucose module 210 is configured to send the generated blood glucose reading (e.g., already in USSD format) to the GSM module 280 via connection 235.

In some embodiments, the blood glucose module 210 may be configured to various test modes for testing blood glucose levels, for example, before meal test mode, after meal test mode, and control solution test mode. The blood glucose module 210 may be configured for single user mode or multi user mode. The electronic blood glucose measurement device 110 may be configured to support menus and alerts in many different languages, for example, up to 80 different languages in one embodiment, enabling the electronic blood measurement device 110 described herein usable in practically any country in the world. In the exemplary embodiment shown in FIG. 2, the electronic blood glucose measurement device 110 also includes a USB port 295, which permits a user to connect a charger cable for charging the electronic blood glucose measurement device 110, or to connect the electronic blood glucose measurement device 110 to a personal computer, for example, to transfer blood glucose readings, or to update the firmware of the electronic blood glucose measurement device 110.

The electronic blood glucose measurement device 110 may optionally include a test strip ejector (e.g., a pushable and/or slidable button) that may be actuated by a user after the blood glucose reading is generated by the blood glucose module 210 in order to eject the test strip from the test strip slot 285. It will be appreciated that when an electronic blood glucose measurement device 110 does not include the optional test strip ejector feature, the user may simply use his or her fingers to remove the test strip from the test strip slot 285 of the electronic blood glucose measurement device 110 after the electronic blood glucose measurement device 110 generates and displays on the display 260 a blood glucose reading based on the test strip. In the embodiment of FIG. 2, the microprocessor of the blood glucose module 210 may be programmed to send the generated blood glucose reading to the GSM module 280 via the connection 235 in direct response to the removal of the test strip from the test strip slot 285.

In some embodiments, the blood glucose module 210 may format the generated blood glucose reading as a USSD message prior to sending the blood glucose reading-containing USSD message to the GSM module 280 via the connection 235 (e.g., UART), such that the GSM module 280 receives a USSD message including the blood glucose reading and transmits the USSD message via the SIM 290 over the network 170 to the central server 130. In other embodiments, the blood glucose module 210 may send the blood glucose reading to the GSM module 280 without formatting it as an USSD message, and the GSM module 280, after receiving the blood glucose reading from the blood glucose module 210, formats the received blood glucose reading to a USSD message format, and transmits this USSD message including one or more blood glucose readings via the SIM module 290 to the central server 130. In some embodiments, the USSD message transmitted from the electronic blood glucose measurement device 110 over the network 170 includes no more than 80 characters, which permits the GSM module 280 to send the USSD message over any of the known telecommunication networks, including hundreds of 2G networks, 3G networks, and/or 4G networks. It will be appreciated that in some embodiments, the USSD message may include up to 140 characters.

Unstructured Supplementary Service Data (USSD) is a GSM network service providing for fast communication between the user and an application or server. USSD is session-oriented technology and data transfer is performed within one session. USSD is advantageous over various other messaging systems since USSD is direct peer-to-peer or point-to-point messaging. One difference between USSD systems and Interactive Voice Response [IVR] systems is that USSD does not involve voice channels to establish connection between service and subscriber terminal. USSD has a number of benefits over short message service (SMS), some of which are described below.

USSD messaging is typically used for communication of proprietary messages between an end user's device and the proprietary services provider. In some embodiments, USSD messages may be up to 140 characters and end with the character #, which indicates end of transmission of the USSD message. In some embodiments, the signaling signal system no. 7 mobile application part (SS7 MAP) protocol treats the USSD message as a proprietary message of the home network. In some embodiments, when a blood glucose measurement device 110 generates and transmits a USSD message, the message is registered on the visitor location register (VLR), and the VLR relays the USSD message directly, without any manipulation to the home location register (HLR), such that the HLR analyzes the USSD message and filters out supplemental service information and relays the remaining message including the blood glucose readings to the carrier USSD server. In some embodiments, the carrier USSD server is configured to analyze the received message, convert it into XML and relay it to the proprietary application server (i.e., central server 130) for processing, thus the central server 130 processes a XML message. In some embodiments, the USSD message is a bi-directional message such that any message received from the electronic blood glucose measurement device 110 will get an uninterrupted feedback from the central server 130.

USSD messaging, as used by the devices and in the systems and methods described herein, has various advantages over short message service (SMS) messaging. For example, USSD is more cost-effective than short message service messaging and permits the sending of messages worldwide, as the USSD messages are compatible with practically all of the available GSM networks. USSD is much faster (e.g., 3-7 times) than SMS messaging, since USSD is session based and establishes an active connection over the network. In some embodiments, from the time a test strip is removed from a test strip slot 285 of the electronic blood glucose measurement device 110 after the blood glucose test is complete to the time an acknowledgement message is displayed on the electronic blood glucose measurement device 110 to indicate that the blood glucose readings have been successfully received and recorded in the central database takes up to about 10 to about 20 seconds, which is significantly faster than a results that would be obtained using an SMS messaging system. In some embodiments, if an error in recording the blood glucose readings to the central database 140 is detected, a USSD message may be sent from the central database 140 to the electronic blood glucose measurement device 110 to reflect that an error has occurred. Notably, sending messaging via USSD is more secure than sending messages via SMS, since USSD is a direct point-to-point communication without rerouting and much more reliable than SMS messaging, where the SMS messages sometimes get lost along the way. A further advantage of USSD over SMS is that SMS uses external aggregators along the way to route the SMS messages, whereas such external aggregators are not needed in USSD messaging systems.

In some embodiments, each USSD message transmitted from the electronic blood glucose measurement device 110 includes up to four blood glucose readings. In one embodiment, the USSD message includes electronic data representing one last (i.e., most recent) successful blood glucose reading successfully transmitted from the electronic blood glucose measurement device 110 to the central server 130 and one new blood glucose reading generated by the electronic blood glucose measurement device 110. The redundant transmission in the USSD message of the most recent blood glucose reading successfully transmitted to the central server 130 may increase the chances of avoiding inadvertent loss of blood glucose reading data unsuccessfully transmitted to the central server 130 over the network 170 due to poor GSM signal or any other reason. In one exemplary embodiment, the USSD message transmitted from the GSM module of the electronic blood glucose measurement device 110 to the central server 130 includes one most recent blood glucose reading successfully transmitted from the electronic blood glucose measurement device 110 to the central server 130, one new blood glucose reading generated by the electronic blood glucose measurement device 110, and two blood glucose readings that were unsuccessfully transmitted to the central server 130 by the electronic blood glucose measurement device 110, and which were stored in the memory 220 of the electronic blood glucose measurement device 110. In another embodiment, the USSD message includes one most recent blood glucose reading successfully transmitted from the electronic blood glucose measurement device 110 to the central server 130 and three blood glucose readings that were unsuccessfully transmitted to the central server 130 by the electronic blood glucose measurement device 110, and which were stored in the memory 220 of the electronic blood glucose measurement device 110.

In some embodiments, after a USSD message including one or more blood glucose readings is transmitted to the central server 130 from the electronic blood glucose measurement device 110, the electronic blood glucose measurement device 110 may receive an acknowledgement signal from the central database 140 via the central server 130 indicating whether the USSD message was successfully received at the central server 130 and recorded in the central database 140. In the exemplary embodiments of FIG. 1, the central database 140 is in communication with the electronic blood glucose measurement device 110 via the central server 130 (through communication pathway 135) and the central server 130 is in turn is in communication with the electronic blood glucose measurement device 110 via the communication pathway 125 (over the network 170). The acknowledgement signal from the central server 130 may be transmitted to the electronic blood glucose measurement device 110 in the form of a USSD message and may be received by the GSM module 280 of the electronic blood glucose measurement device 110.

In some embodiments, the electronic blood glucose measurement device 110, after receiving an indication from the central server 130 or the central database 140 (e.g., error message) that the USSD message was not successfully transmitted from the electronic blood glucose measurement device 110 to the central server 130, may store the last unsuccessfully transmitted blood glucose readings in the non-volatile memory 220. Then, after a new blood glucose reading is generated by the blood glucose module 210, the electronic blood glucose measurement device 110 may transmit a USSD message including the stored unsuccessfully transmitted blood glucose reading, the last successfully transmitted blood glucose reading (for synchronization purposes), and the newly-generated blood glucose reading to the central server 130 when a connection to the central server 130 over the network 170 becomes available. The non-volatile memory 220 may store dozens (or hundreds) of the unsuccessfully transmitted blood glucose readings. For example, up to 10 unsuccessfully transmitted blood glucose readings may be stored in the non-volatile memory 220 in one embodiment, up to 15 unsuccessfully transmitted blood glucose readings may be stored in the non-volatile memory 220 in another embodiment, up to 20 or more unsuccessfully transmitted blood glucose readings may be stored in the non-volatile memory 220 in another embodiment, and up to 100 or more unsuccessfully transmitted blood glucose readings may be stored in the non-volatile memory 220 in another embodiment.

The electronic blood glucose measurement device 110 may use one or more data pointers to refer to the blood glucose readings stored in the non-volatile memory 220. In some embodiments, an indication pointer may point to an address in the non-volatile memory 220 representing a last blood glucose reading successfully transmitted from the electronic blood glucose measurement device 110 to the central server 130 over the network 170. In some embodiments, after the electronic blood glucose measurement device 110 receives a confirmation (e.g., acknowledgement USSD message) from the central server 130 indicating that the USSD message transmitted from the electronic blood glucose measurement device 110 over the network 170 was successfully received by the central server 130, the electronic blood glucose measurement device 110 updates the indication pointer to point to an address in the non-volatile memory 220 representing the latest blood glucose reading successfully transmitted to the central server 130.

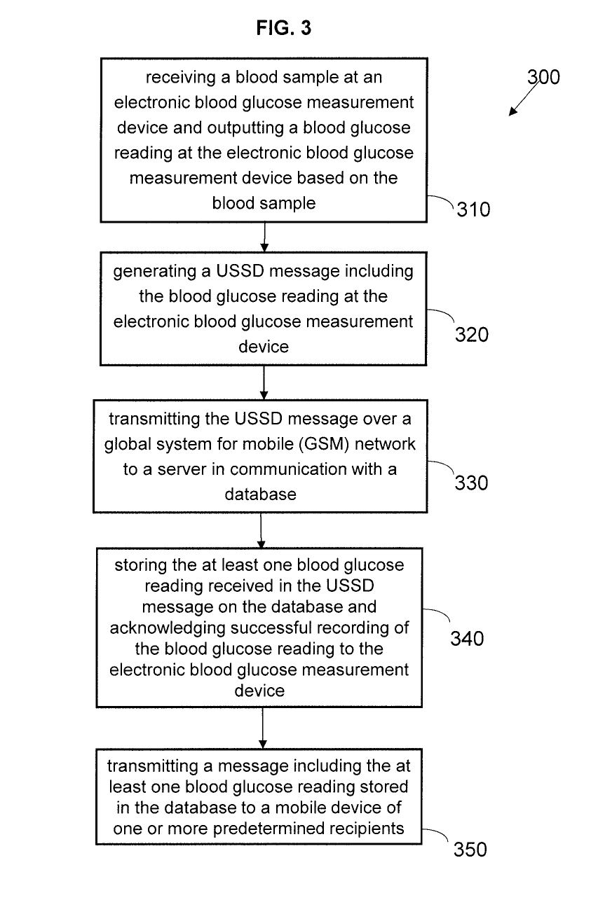

With reference to FIGS. 1-3, one method 300 of operation of the system 100 to transmit blood glucose reading data over a network will now be described. For exemplary purposes, the method 300 is described in the context of the system of FIG. 1, but it is understood that embodiments of the method 300 may be implemented in the system 100 or other systems. Generally, as shown in FIG. 3, the method 300 includes receiving a blood sample at an electronic blood glucose measurement device 110 and outputting at least one blood glucose reading at the electronic blood glucose measurement device 110 based on the blood sample (step 310).

After the blood glucose reading is generated (e.g., by the blood glucose module 210 of the electronic blood glucose measurement device 110), the electronic blood glucose measurement device 110 generates a USSD message including at least the newly-generated blood glucose reading (step 320). As discussed above, in addition to the newly-generated blood glucose reading, the USSD message may include the last blood glucose reading successfully transmitted from the electronic blood glucose measurement device 110 to the central server 130, and the USSD message may be generated by the blood glucose module 210 or by the GSM module 280.

After the USSD message is generated, the electronic blood glucose measurement device 110 transmits the USSD message from the GSM module 180 via the SIM 290 over a GSM network 170 to the central server 130, which is in communication with a central database 140 (step 330). The blood glucose readings received in the USSD message from the electronic blood glucose measurement device 110 are then stored on the central database 140 (step 340). As discussed above, the central database 140 may be a hard drive internal to or external to the central server 130, may be at a physical site shared with the central server 130, or may be at a location remote to (e.g., thousands of miles away from) the central server 130, and may be in the form of a website having an IP address, such that each blood glucose reading is stored in the central database 140 with its own unique device ID.

The exemplary method shown in FIG. 3 includes the optional step of transmitting a message including the at least one blood glucose reading from the central database 140 to a personal electronic (e.g. mobile) device 150 of one or more predetermined recipients (step 350). As discussed above, the transmission of the blood glucose reading stored in the central database 140 to the personal electronic device 150 is not only optional to the method depicted in FIG. 3, but is optional to the exemplary system 100 shown in FIG. 1. In other words, the communication channel 145 over the network 180 in FIG. 1 is optional and the system 100 may not include an active communication channel that permits the glucose readings stored in the central database 140 to be sent to the personal electronic device 150 via the central server 130. Instead, in some embodiments, one or more network devices or servers not including the central server 130 may be utilized to provide a communication channel through which the blood glucose readings may be transmitted from the central database 140 to one or more personal electronic devices 150. In some optional embodiments, the message including the at least one blood glucose reading from the database 140 may be sent from the central server 130 over the network 180 to a personal electronic device 150 of the recipient via short message service (SMS) or another messaging service (e.g., instant messenger, electronic mail, push notification to a mobile app, or the like). In some optional embodiments, the central database 140 may be in website format and may permit for the messages sent to the personal electronic device 150 to include various information relating to the blood glucose readings, for example, reports, trends, graphs, and alerts, just to name a few. In the exemplary embodiment of FIG. 1, the central database 140 is in communication with the personal electronic device 150 via the central server 130 (through communication pathway 135), and the central server 130 is in turn is in communication with the personal electronic device 150 via the communication pathway 145 (over the network 180).

In some embodiments, the GSM network 170 may include a home location register (HLR) configured to receive the USSD message transmitted from the electronic blood glucose measurement device 110. In one embodiment, the HLR sends the USSD message to a USSD server and the USSD server converts the message into XML format and transmits the XML command to an IP address of a website-based central database 140. Then, the central database 140, after receiving the XML command from the central server 130, processes the XML command to store the received blood glucose readings and to generate a reply XML command and transmits the reply XML command to the central server 130. Then, the central server 130, after receiving the reply XML command from the central database 140, transmits the reply XML command received from the database to the USSD server which convert it to USSD message and sends it to the HLR. The HLR then sends the USSD message to the electronic blood glucose measurement device 110 (e.g., to indicate to the electronic blood glucose measurement device 110 that the USSD message including the most recently transmitted blood glucose readings successfully reaches the central server 130).

Some exemplary messages that may be sent by/between the components of the system 100 of FIG. 1 during the exemplary process of FIG. 3 are as follows:

Message sent to central database 140:

<mo-ussd-submit-request version=“1.0” id=“791”>

<msisdn>19038904313</msisdn>
<user-data>U0000800110824070213470000000,
110829160077777000000#</user-data>
<imsi>310630803000351</imsi>

</mo-ussd-submit-request>

Then, upon successful processing at the central database 140, the central database 140 may return the following message to the central sever 130:

<mo-ussd-submit-response>

<user-data>successx</user-data>

</mo-ussd-submit-response>

In some embodiments, the central database 140 replies by “echo” at the end of the script which handles the transaction as follows: echo ‘<mo-ussd-submit-response><user-data>successx</user-data></mo-ussd-submit-response>’

If, for some reason, the central database fails to process the blood glucose reading data received from the central server 130, the central database 140 may return the following message to the central server 130:

<mo-ussd-submit-response>

<user-data>Invalid argument supplied</user-data>

</mo-ussd-submit-response>

Then, an exemplary message that may be returned from the central server 130 to the electronic blood glucose measurement device 110 may be as follows:

<mo-ussd-submit-response>

<user-data>successx</user-data> or <user-data>Invalid argument
supplied</user-data>

</mo-ussd-submit-response>

The devices, systems, and methods, described in the present application advantageously utilize USSD messages to transmit blood glucose readings over a network to a database. The utilization of the USSD messages by the devices, systems, and methods described herein provide a faster, simpler, more secure, and more cost-efficient way to send blood glucose readings from electronic blood glucose measurement devices over a network than conventional messenger platforms such as short message service (SMS).

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. An electronic blood glucose measurement device comprising:
a blood glucose module including a microprocessor;
a global system for mobile (GSM) module in communication with the blood glucose module; and
a subscriber identification module (SIM) in communication with the GSM module;
wherein the GSM module is configured to:
receive a blood glucose reading from the blood glucose module;
connect to a network via the SIM; and
transmit an Unstructured Supplementary Service Data (USSD) message including the blood glucose reading over a network to a server in communication with a database configured to store the blood glucose reading.

2. The device of claim 1, further comprising an input configured to receive a test strip, wherein the blood glucose module is configured to generate a blood glucose reading based on a blood sample applied to the test strip and to send the generated blood glucose reading to the GSM module in response to removal of the test strip from the input.

3. The device of claim 1 wherein at least one of the microprocessor of the blood glucose module and the GSM module are configured to format the at least on blood glucose reading in order to generate the USSD message.

4. The device of claim 1, wherein the SIM is one of a physical SIM card and a virtual SIM

5. The device of claim 1, wherein the USSD message is up to 80 characters.

6. The device of claim 1, wherein the at least one blood glucose reading in the USSD message includes a reading representing a last blood glucose reading successfully transmitted to the server and the blood glucose reading.

7. The device of claim 1, further comprising a non-volatile memory and an indication pointer configured to point to an address in the non-volatile memory representing a last blood glucose reading successfully transmitted to the server.

8. The device of claim 7, wherein the device is configured to receive a confirmation from the server indicating that the USSD message transmitted from the device was received by the server, and to update the indication pointer to point to an address in the non-volatile memory representing the at least one blood glucose reading successfully transmitted to the server.

9. The device of claim 7, wherein the device is configured, upon receiving an indication that the USSD message was not successfully transmitted from the device to the server, to store the blood glucose reading in the non-volatile memory, and to transmit another USSD message including the stored blood glucose reading after another blood glucose reading is generated by the blood glucose module when a connection to the server becomes available.

10. The device of claim 8, wherein the device is configured to receive the confirmation from the server in a form of an USSD message.

11. A system for transmitting blood glucose readings, the system comprising:
an electronic blood glucose measurement device; and
a server in communication with the electronic blood glucose measurement device over a global system for mobile communications (GSM) network;
wherein the electronic blood glucose measurement device is configured to generate a blood glucose reading and transmit an Unstructured Supplementary Service Data (USSD) message including the blood glucose reading to the server over the GSM network.

12. The system of claim 11, further comprising a home location register (HLR) configured to receive the USSD message transmitted from the electronic blood glucose measurement device, and wherein the server is configured to receive a command representing the USSD message converted to an Extensible Markup Language (XML) command.

13. The system of claim 12, wherein the server is configured to transmit the XML command to an IP address of a website-based database.

14. The system of claim 13, wherein the database is configured to process the XML command and store the received blood glucose reading, to generate a reply XML command, and transmit the reply XML command to the server.

15. The system of claim 14, wherein the server is configured to transmit the reply XML command received from the database to the HLR.

16. The system of claim 15, wherein the electronic blood glucose measurement device is configured to receive a message representing the reply XML converted to a reply USSD message.

17. The system of claim 16, wherein the electronic blood glucose measurement device includes a visual display configured to display a message indicating whether the USSD message was successfully transmitted from the electronic blood glucose measurement device to the server and successfully stored in the database.

18. The device of claim 1, wherein the electronic blood glucose measurement device further includes a GSM module and a subscriber identification module (SIM), and wherein the electronic blood glucose measurement device is configured to transmit the USSD message to the server from the GSM module via the SIM.

19. The system of claim 11, wherein the database is configured to send a text message including the blood glucose reading to a mobile device of one or more predetermined recipients.

20. A method of transmitting a blood glucose reading, the method comprising:
receiving a blood sample at an electronic blood glucose measurement device and outputting a blood glucose reading at the electronic blood glucose measurement device based on the blood sample;
generating an Unstructured Supplementary Service Data (USSD) message including the blood glucose reading at the electronic blood glucose measurement device;
transmitting the USSD message over a global system for mobile (GSM) network to a server in communication with a database; and
storing the blood glucose reading received in the USSD message on the database and acknowledging successful recording of the blood glucose reading to the electronic blood glucose measurement device.

21. The method of claim 20, further comprising transmitting a message including the blood glucose reading stored in the database to a mobile device of one or more predetermined recipients.
